(19) United States
(12) Patent Application Publication
(10) Pub. No.: US 2025/0064609 A1
(43) Pub. Date: Feb. 27, 2025

(54) STENT WITH PLAQUE CAPTURE MESH

(71) Applicant: Boston Scientific Scimed, Inc., Maple Grove, MN (US)

(72) Inventors: Aiden Flanagan, Kilcolgan / Co. Galway (IE); Cass Alexander Hanson, St. Paul, MN (US)

(73) Assignee: Boston Scientific Scimed, Inc., Maple Grove, MN (US)

(21) Appl. No.: 18/811,097

(22) Filed: Aug. 21, 2024

Related U.S. Application Data
(60) Provisional application No. 63/534,616, filed on Aug. 25, 2023.

(51) Int. Cl. A61F 2/90; A61F 2/00; A61F 2/01

(52) U.S. Cl. CPC A61F 2/90; A61F 2002/0081; A61F 2002/016

(57) ABSTRACT

A medical stent includes an expandable structure that is moveable between a collapsed configuration for delivery and an expanded configuration for deployment, the expandable structure in the expanded configuration defining a relaxed diameter. The expandable structure retains its relaxed diameter along portions of the expandable structure that are not constrained by contact with vessel walls after deployment. Portions of the expandable structure that are compressed by contact with vessel walls after deployment are caused to move to a reduced diameter less than the relaxed diameter along portions. The compressed portions of the expandable structure may help prevent plaque from breaking loose. Uncompressed portions of the expandable structure may allow blood flow therethrough.

### CROSS-REFERENCE TO RELATED APPLICATIONS

The present application claims the benefit of U.S. Provisional Patent Application Ser. No. 63/534,616, filed Aug. 25, 2023, the disclosure of which is incorporated herein by reference.

### TECHNICAL FIELD

The present disclosure relates generally to methods and apparatuses for various digestive ailments. More particularly, the disclosure relates to different configurations and methods of manufacture and use of a stent.

### BACKGROUND

Implantable stents are devices that are placed in a body structure, such as a blood vessel, esophagus, trachea, biliary tract, colon, intestine, stomach or body cavity, to provide support and to maintain patency of the structure. These devices are manufactured by any one of a variety of different manufacturing methods and may be used according to any one of a variety of methods for a variety of applications. Of the known medical devices, delivery systems, and methods, each has certain advantages and disadvantages. There is an ongoing need to provide alternative medical devices and delivery devices as well as alternative methods for manufacturing and using medical devices and delivery devices.

### SUMMARY

The disclosure is directed to several alternative designs, materials and methods of manufacturing medical device structures and assemblies, and the use thereof.

An example may be found in a medical stent. The medical stent includes an expandable structure moveable between a collapsed configuration for delivery and an expanded configuration for deployment, the expandable structure in the expanded configuration defining a relaxed outer diameter. The expandable structure retains its relaxed outer diameter along portions of the expandable structure that are not constrained by contact with vessel walls after deployment. The expandable structure moves to a reduced outer diameter less than the relaxed outer diameter along portions of the expandable structure that are compressed by contact with vessel walls after deployment.

Alternatively or additionally, the expandable structure may define a plurality of voids through a wall of the expandable structure, and the voids have a first average size when the expandable structure retains its relaxed configuration, and the voids have a second average size less than the first average size when the expandable structure is compressed to the reduced outer diameter.

Alternatively or additionally, the expandable structure may include an inner layer and an outer layer moveable relative to the inner layer, and where the outer layer is spaced a first distance from the inner layer when the expandable structure retains its relaxed outer diameter, and the outer layer is spaced a second distance from the inner layer when the expandable structure is compressed to its reduced outer diameter. The second distance is less than the first distance.

Alternatively or additionally, the expandable structure may include a knitted stent.

Alternatively or additionally, the knitted stent may include a plurality of loops, at least some of which have a relaxed configuration in which the loops extend radially outwardly relative to a longitudinal axis of the medical stent.

Alternatively or additionally, the loops that extend radially outwardly relative to the longitudinal axis of the medical stent may be adapted to fold over in response to being compressed by contact with the vessel wall.

Alternatively or additionally, the loops having a relaxed configuration in which the loops extend radially outwardly may define the relaxed outer diameter of the expandable structure.

Alternatively or additionally, the loops that are adapted to fold over in response to being compressed may define the reduced outer diameter of the expandable structure when the loops are folded over.

Alternatively or additionally, the expandable structure may include one or more elongated struts and one or more filaments wrapped around each of the one or more elongated struts. The one or more filaments form coils having a relaxed configuration defining the relaxed outer diameter of the expandable structure, and portions of the coils that are compressed from contact with the vessel wall define the reduced outer diameter of the expandable structure.

Another example may be found in a medical stent. The medical stent includes a tubular substrate extending from a proximal end to a distal end, the tubular substrate moveable between a radially collapsed configuration and a radially expanded configuration, and a compliant layer extending over at least part of the tubular substrate, the compliant layer having a relaxed thickness when not radially constrained, the compliant layer adapted to radially compress into a reduced thickness when radially constrained.

Alternatively or additionally, the compliant layer may include a plurality of loops, at least some of which have a relaxed configuration in which the loops extend radially outwardly relative to a longitudinal axis of the medical stent.

Alternatively or additionally, the loops that extend radially outwardly relative to the longitudinal axis of the medical stent may be adapted to fold over in response to being compressed by contact with the vessel wall.

Alternatively or additionally, the loops having a relaxed configuration in which the loops extend radially outwardly may define the relaxed thickness of the compliant layer.

Alternatively or additionally, the loops that are adapted to fold over in response to being compressed may define the reduced thickness of the compliant layer when the loops are folded over.

Another example may be found in a medical stent that is adapted for deployment within a blood vessel having a side branch extending from the blood vessel, the medical stent adapted to be deployed within the blood vessel at a position in which the medical stent spans the side branch. The medical stent includes a substrate extending from a proximal end to a distal end, the substrate defining a tubular body moveable between a radially collapsed configuration and a radially expanded configuration, and a compliant layer extending over at least part of the substrate, the compliant layer adapted to being radially compressed by contact with a vessel wall of the blood vessel. A first portion of the compliant layer is configured to be radially compressed by contact with the vessel wall when deployed within the blood vessel, and a second portion of the compliant layer corresponding to where the side branch extends is configured to not be radially compressed or be compresses to a lesser degree than the first portion when deployed within the blood vessel.

Another example may be found in a medical stent. The medical stent includes an expandable structure moveable between a collapsed configuration for delivery and an expanded configuration for deployment, the expandable structure in the expanded configuration defining a relaxed outer diameter. The expandable structure retains its relaxed outer diameter along portions of the expandable structure that are not constrained by contact with vessel walls after deployment, and the expandable structure moves to a reduced outer diameter less than the relaxed outer diameter along portions of the expandable structure that are compressed by contact with vessel walls after deployment.

Alternatively or additionally, the expandable structure defines a plurality of voids through a wall of the expandable structure. The voids have a first average size when the expandable structure retains its relaxed configuration, and the voids have a second average size less than the first average size when the expandable structure is compressed to the reduced outer diameter.

Alternatively or additionally, the expandable structure may include an inner layer and an outer layer moveable relative to the inner layer to vary a thickness of a wall of the expandable structure. The outer layer is spaced a first distance from the inner layer when the expandable structure retains its relaxed outer diameter; and the outer layer is spaced a second distance from the inner layer when the expandable structure is compressed to its reduced outer diameter. The second distance is less than the first distance.

Alternatively or additionally, the expandable structure may include a knitted stent.

Alternatively or additionally, the knitted stent may include a plurality of loops, at least some of which have a relaxed configuration in which the loops extend radially outwardly relative to a longitudinal axis of the medical stent.

Alternatively or additionally, the loops that extend radially outwardly relative to the longitudinal axis of the medical stent may be adapted to fold over in response to being compressed by contact with the vessel wall, thereby reducing the wall thickness of the expandable structure.

Alternatively or additionally, the loops having a relaxed configuration in which the loops extend radially outwardly may define the relaxed outer diameter of the expandable structure.

Alternatively or additionally, the loops that are adapted to fold over in response to being compressed may define the reduced outer diameter of the expandable structure when the loops are folded over, thereby reducing the wall thickness of the expandable structure.

Alternatively or additionally, the expandable structure may include one or more elongated struts and one or more filaments wrapped around each of the one or more elongated struts. The one or more filaments form coils having a relaxed configuration defining the relaxed outer diameter of the expandable structure. Portions of the coils that are compressed from contact with the vessel wall define the reduced outer diameter of the expandable structure thereby reducing the wall thickness of the expandable structure.

Another example may be found in a medical stent. The medical stent includes a tubular substrate extending from a proximal end to a distal end, the tubular substrate moveable between a collapsed configuration and an expanded configuration, and a compliant layer extending over at least part of the tubular substrate, the compliant layer having a relaxed thickness when not constrained, the compliant layer adapted to compress into a reduced thickness when constrained.

Alternatively or additionally, the compliant layer defines a plurality of voids. The voids have a first average size when the compliant layer retains its relaxed configuration, and the voids have a second average size less than the first average size when the compliant layer is compressed to the reduced thickness.

Alternatively or additionally, the tubular substrate may include an inner layer and the compliant layer may include an outer layer moveable relative to the inner layer. The outer layer may be spaced a first distance from the inner layer when the compliant layer retains its relaxed thickness, and the outer layer may be spaced a second distance from the inner layer when the compliant layer is compressed to its reduced thickness. The second thickness is less than the first thickness.

Alternatively or additionally, the compliant layer may include a plurality of loops, at least some of which have a relaxed configuration in which the loops extend radially outwardly relative to a longitudinal axis of the medical stent.

Alternatively or additionally, the loops that extend radially outwardly relative to the longitudinal axis of the medical stent may be adapted to fold over in response to being compressed by contact with the vessel wall, thereby reducing the wall thickness of the expandable structure.

Alternatively or additionally, the loops having a relaxed configuration in which the loops extend radially outwardly may define the relaxed thickness of the compliant layer.

Alternatively or additionally, the loops that are adapted to fold over in response to being compressed may define the reduced thickness of the compliant layer when the loops are folded over.

Another example may be found in a medical stent adapted for deployment within a blood vessel having a side branch extending from the blood vessel, the medical stent adapted to be deployed within the blood vessel at a position in which the medical stent spans the side branch. The medical stent includes a substrate extending from a proximal end to a distal end, the substrate defining a tubular body moveable between a collapsed configuration and an expanded configuration, and a compliant layer extending over at least part of the substrate, the compliant layer adapted to being compressed by contact with a vessel wall of the blood vessel. A portion of the compliant layer corresponding to where the side branch extends from the blood vessel remains uncompressed.

Alternatively or additionally, the compressed portion of the compliant layer may have a smaller void size than that of the uncompressed portion of the compliant layer.

Alternatively or additionally, the compressed portion of the compliant layer may hold plaque in place between the compliant layer and the vessel wall.

Alternatively or additionally, the uncompressed portion of the compliant layer may allow blood to flow through the compliant layer between the blood vessel and the side branch.

The preceding summary is provided to facilitate an understanding of some of the innovative features unique to the present disclosure and is not intended to be a full description. A full appreciation of the disclosure can be gained by taking the entire specification, claims, figures, and abstract as a whole.

### BRIEF DESCRIPTION OF THE DRAWINGS

The disclosure may be more completely understood in consideration of the following description of various examples in connection with the accompanying drawings, in which:

FIG. 1 is a partial cutaway view of a portion of a human head and neck, illustrating some of the vasculature within the neck;

FIG. 2 is a schematic view of an example medical stent that includes an inner tubular member and an outer tubular member;

FIG. 3 is a schematic view of an example medical stent deployed within a bifurcated or branched blood vessel;

FIG. 4 is a cross-sectional view taken along the line 4-4 of FIG. 3;

FIG. 5 is a cross-sectional view taken along the line 5-5 of FIG. 3;

FIGS. 6A, 6B and 6C are schematic views of a portion of an example stent;

FIGS. 7A and 7B are schematic views of a portion of an example stent;

FIG. 8 is a schematic view of a portion of an example stent;

FIGS. 9A and 9B are schematic views of a portion of an example stent

FIG. 10 is a schematic view of a portion of an example stent, showing an open loop knitting pattern;

FIG. 11 is a schematic view of a portion of an example stent, showing a twisted loop knitting pattern; and

FIGS. 12A and 12B are schematic views of a portion of an example stent.

While the disclosure is amenable to various modifications and alternative forms, specifics thereof have been shown by way of example in the drawings and will be described in detail. It should be understood, however, that the intention is not to limit the disclosure to the particular examples described. On the contrary, the intention is to cover all modifications, equivalents, and alternatives falling within the spirit and scope of the disclosure.

### DESCRIPTION

The following description should be read with reference to the drawings, in which like elements in different drawings are numbered in like fashion. The drawings, which are not necessarily to scale, depict examples that are not intended to limit the scope of the disclosure. Although examples are illustrated for the various elements, those skilled in the art will recognize that many of the examples provided have suitable alternatives that may be utilized.

All numbers are herein assumed to be modified by the term “about”, unless the content clearly dictates otherwise. The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is contemplated that the feature, structure, or characteristic may be applied to other embodiments whether or not explicitly described unless clearly stated to the contrary.

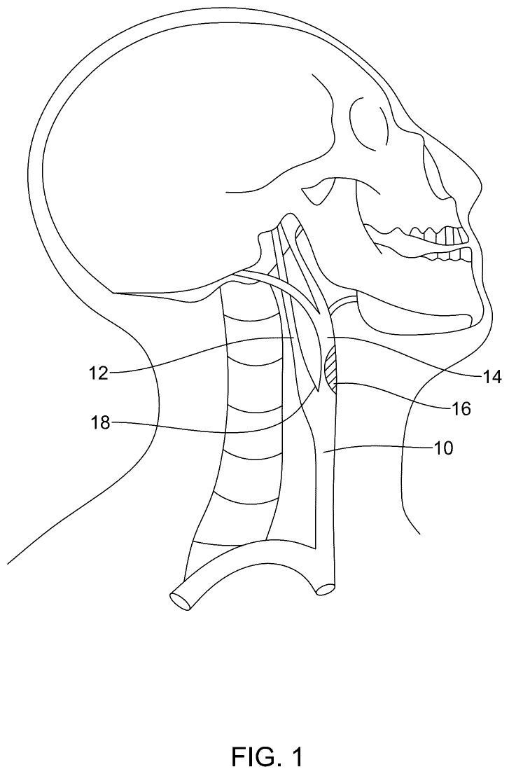

FIG. 1 is a partial cut-away view of the human head and neck, showing some of the vasculature. FIG. 1 shows a common carotid artery 10, which bifurcates into the external carotid artery 12 and the internal carotid artery 14. This is just one of a number of examples of blood vessels that bifurcate, or have side branches. A lesion 16 is schematically shown within the internal carotid artery 14, just above a bifurcation point 18. An arterial sheath may be advanced up through the vasculature to a point within the common carotid artery 10 and an inflatable occlusion balloon carried by the arterial sheath may be used to occlude anterograde blood flow through the common carotid artery 10. The common carotid artery 10 may be reached by advancing through the arterial system to the common carotid artery 10. The arterial system may be accessed via a number of different arteries, but in some instances, the arterial system may be accessed via one of the patient's femoral arteries, as the patient has a femoral artery extending through the groin and down either leg. In some instances, other arteries providing a shorter path to the common carotid artery 10 may be utilized. A variety of different procedures may be performed once the common carotid artery has been accessed.

In some instances, a stent may be deployed within the common carotid artery 10, and in some instances the stent may overly the external carotid artery 14, as an example. Placing the stent in this location may cause plaque that is disposed on the vessel walls of the common carotid artery 10 to be dislodged. There is a desire to minimize dislodgement of plaque, particularly in this area, as dislodged plaque may travel to the brain, particularly if the plaque reaches the internal carotid artery 12. Moreover, there are multiple vascular connections between the internal carotid artery 12 and the external carotid artery 14 within the head. When the stent overlies the external carotid artery 14, the stent itself may limit blood flow from the common carotid artery 10 to the external carotid artery 14. In some instances, there is a desire to allow this blood flow without materially affecting this blood flow.

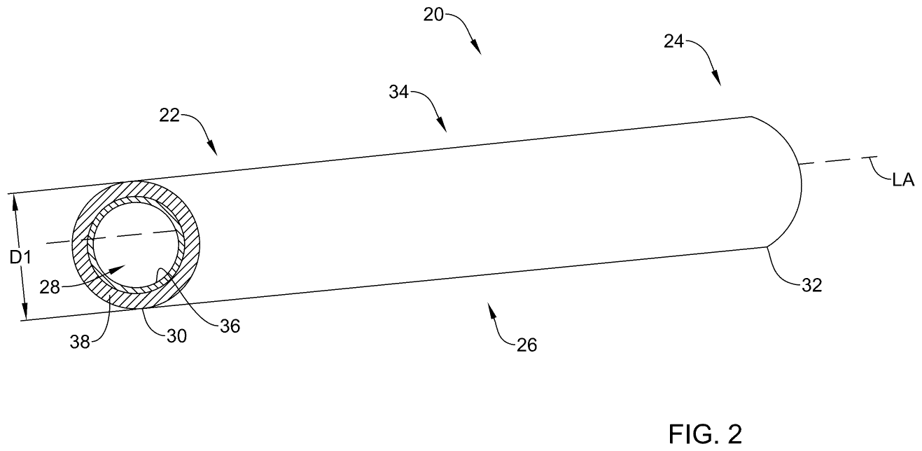

FIG. 2 is a schematic view of an example medical stent 20. In FIG. 2, while the example medical stent 20 is shown as generally tubular, it is contemplated that the medical stent 20 may take any cross-sectional shape desired. The medical stent 20 may be considered as including a proximal end region 22, a distal end region 24, and an intervening intermediate region 26, recognizing that these designations are arbitrary, and depend on the orientation in which the medical stent 20 will ultimately be implanted within a body lumen. The medical stent 20 may be considered as having a constant diameter throughout, including through the proximal end region 22, the distal end region 24 and the intervening intermediate region 26, particularly when unconstrained. When the medical stent 20 is deployed within a blood vessel, it will be appreciated that the blood vessel will likely impact the profile of the medical stent 10 as the blood vessel may cause parts of the medical stent 20 to become compressed or otherwise deformed. In some instances, the medical stent 20 may be considered as having a constant diameter when in a relaxed. equilibrium state. The medical stent 20 may include a lumen 28 that extends from a proximal end 30 of the medical stent 20 to a distal end 32 of the medical stent 20 to allow for the passage of blood therethrough. The medical stent 20 may be considered as having a longitudinal axis LA. The medical stent 20 may be expandable from a first radially collapsed configuration (not shown) to a second radially expanded configuration, which may correspond to its relaxed, equilibrium state. In some instances, the medical stent 20 may be structured to extend across a stricture and to apply a radially outward pressure to the stricture in a lumen in order to open the lumen and allow for the passage of blood therethrough.

The medical stent 20 includes an expandable tubular structure 34 extending from the proximal end 30 to the distal end 32. In some instances, the expandable tubular structure 34 may include a single layer or structure. In some instances, the expandable tubular structure 34 may include a tubular substrate 36 and a compliant layer 38 that it disposed over the tubular substrate 36. In some instances, the expandable tubular structure 34 may be moveable between a radially collapsed configuration (not shown) and a radially expanded configuration (as shown). The tubular substrate 36 may take a variety of forms. In some instances, the tubular substrate 36 may be a stent formed of a plurality of interconnected struts formed as a monolithic structure from a tubular member. For example, the tubular substrate 36 may be a laser-cut stent, which is a stent that is laser-cut from a cylinder, such as a metal tube. The EPIC™ stents made by Boston Scientific, Corporation are examples of laser-cut stents. In other instances, the tubular substrate 36 may be a tubular structure formed of one or more, or a plurality of interwoven wires. In some instances, the tubular substrate 36 may be a braided stent formed of a plurality of braided wires, for example. Some exemplary stents including braided filaments include the WallFlex®, WALLSTENT®, and Polyflex® stents, made and distributed by Boston Scientific, Corporation. In some instances, the tubular substrate 36 may be adapted to provide additional surface for an elutable drug coating, for example. In some instances, the tubular substrate 36 may be a woven tubular member, such as a knitted stent, which is a stent formed by knitting one or more filaments together. In some instances, a knitted stent may include an open loop knitting pattern. In some instances, a knitted stent may include a twisted loop knitting pattern. The Ultraflex™ stents made by Boston Scientific, Corporation are an example of a knitted stent. In some instances, the tubular substrate 36 may be of a knotted type, such the Precision Colonic™ stents made by Boston Scientific, Corporation.

The compliant layer 38, which circumferentially surrounds the tubular substrate 36, may be adapted to have a default or relaxed (e.g., equilibrium) configuration in which the compliant layer 38 maintains a relaxed outer diameter, indicated in FIG. 2 as a diameter D₁. In some instances, portions of the compliant layer 38 may be adapted to compress, or otherwise reduce in outer diameter, in response to portions of the compliant layer 38 being radially constrained by contact with a blood vessel wall, or in response to portions of the compliant layer 38 contacting an obstruction within a blood vessel (such as the lesion 16 shown in FIG. 1). In some instances, the compliant layer 38 may define a first pore or void size when the compliant layer 38 is in its relaxed configuration, at its relaxed diameter D₁, and the compliant layer 38, or portions of the complaint layer 38, may define a second pore or void size when the compliant layer 38 or portions thereof are in radial compression as a result of being radially constrained. The second pore or void size may be smaller than the first pore or void size. As a result, portions of the compliant layer 38 that are compressed as a result of being constrained have a smaller pore or void size and thus may be better at trapping plaque and other debris that may otherwise be released as a result of the medical stent 20 being deployed. Further, portions of the compliant layer 38 that remain in the relaxed configuration, such as where the medical stent 20 spans a side vessel and no radially inward force is applied to the compliant layer 38, have a relatively larger pore or void size, and thus may permit blood to flow between the blood vessel and its side vessel through the sidewall of the stent 20. The medical stent 20 may be considered as an uncovered stent having passageways extending through the tubular wall of the medical stent 20 from an exterior of the medical stent 20 into the lumen of the medical stent 20. The passageways may be defined by the interconnected void spaces of the compliant layer 38 and void spaces of the tubular substrate 36.

In some instances, the compliant layer 38 may be formed of any of a variety of different materials. Depending on the materials and construction of the compliant layer 38, it is contemplated that in some cases the medical stent 20 may include only the compliant layer 38, and may not include the tubular substrate 36. As an example, in some instances, as will be discussed, the compliant layer 38 may include a knitted pattern having a plurality of loops knitted from a filament. At least some of the plurality of loops may extend radially outwardly from the longitudinal axis LA and thus may define the relaxed configuration defining the diameter D₁, while others of the plurality of loops may define the necessary structure to support the compliant layer 38. In some instances, the compliant layer 38 may include filaments that are wound into loose coils, for example.

In some instances, the medical stent 20 may be a self-expanding stent (SES), meaning that the medical stent 20 will automatically expand into its expanded configuration once any constraints preventing expansion have been removed. In some instances, the medical stent 20 may not be a self-expanding stent, and thus may rely upon an inflatable balloon or other expandable member within the lumen 28 in order to cause the medical stent 20 to expand from its collapsed configuration for delivery to its expanded configuration for deployment.

The tubular support 36 and the compliant layer 38 may be formed of any suitable material. For example, the tubular support 36 and the compliant layer 38 may each independently be formed of metals, metal alloys, shape memory alloys, polymers, as desired, enabling the medical stent 20 to be expanded into shape when accurately positioned within the body. In some instances, the material may be selected to enable the medical stent 20 to be removed with relative case as well. For example, the tubular support 36 and the compliant layer 38 may each be formed from alloys such as, but not limited to, nitinol and Elgiloy®. In some instances, one or more of the tubular support 36 and the compliant layer 38 may be formed from one or more nitinol filaments. In some instances, the tubular support 36 may be laser-cut from a nitinol cylinder or tube. In some instances, the combination of the tubular support 36 and the compliant layer 38 may enhance transfer of any elutable drug that is disposed on at least one of the tubular support 36 and the compliant layer 38.

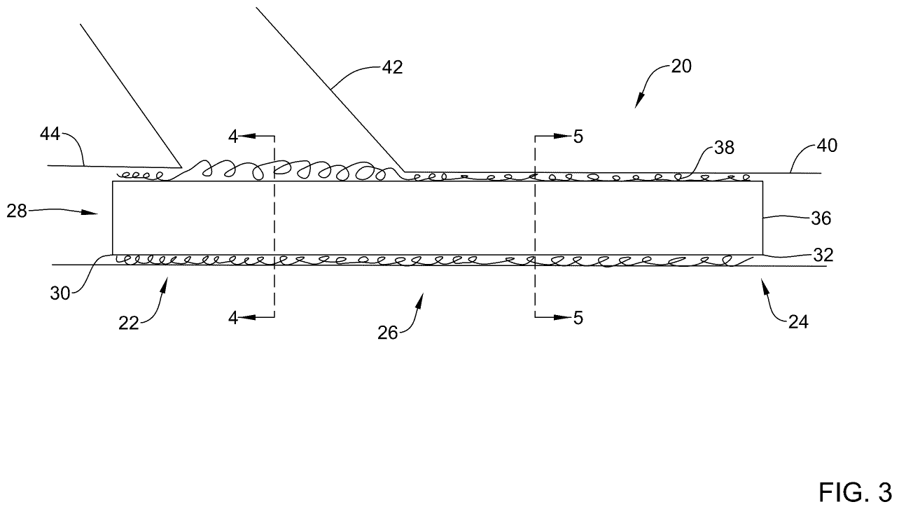

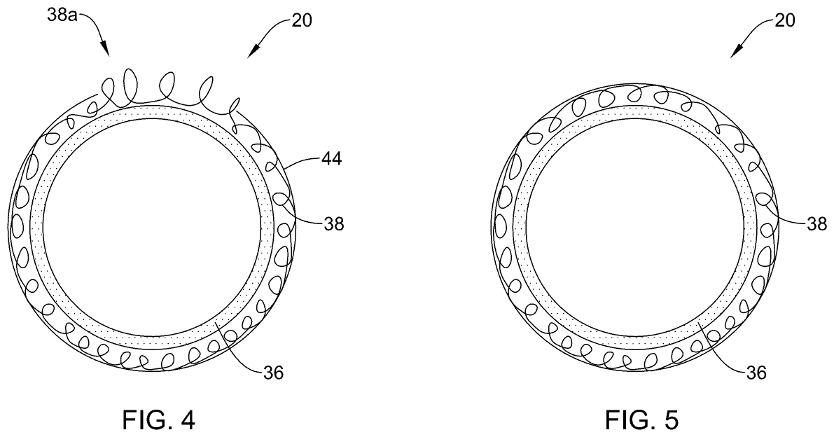

FIG. 3 is a schematic view of the medical stent 20 shown disposed within a blood vessel 40 having a side branch 42 diverging therefrom. The blood vessel 40 includes a vessel wall 44. The medical stent 20 is dimensioned to fit within the blood vessel 40 such that the compliant layer 38 is at least partially radially compressed as a result of being constrained by the vessel wall 44 when the medical stent 20 is radially expanded to its radially expanded configuration. As can be seen, the compliant layer 38 is at least partially radially compressed anywhere the compliant layer 38 contacts the vessel wall 44, and is not radially compressed where the compliant layer 38 extends over the side branch 42 and thus is not constrained by contact with the vessel wall 44. FIG. 4 is a cross-sectional view taken along the line 4-4 and FIG. 5 is a cross-sectional view taken along the line 5-5. As can be seen in FIG. 4, the portion of the compliant layer 38 that is constrained by the vessel wall 44 is compressed into a reduced diameter, thereby resulting in a reduced pore or void size within the compliant layer 38. A portion 38a of the compliant layer 38, overlying the side branch 42 is not compressed, and instead remains at its relaxed diameter. As can be seen in FIG. 5, an entirety of the circumference of the compliant layer 38 is constrained by the vessel wall 44 and thus is compressed into a reduced diameter. Put another way, portions of the compliant layer 38 may have a first, or relaxed, radial thickness (measured from a radial inner extent of the compliant layer 38 to a radially outer extent of the compliant layer 38) when not constrained, and may have a second, or reduced, thickness that is less than the first, or relaxed, thickness, when radially constrained. Thus, as shown in FIG. 4, the portion of the compliant layer 38 that is not contacting the vessel wall 44, and thus not constrained by contact with the vessel wall 44, may have a radial thickness greater than portions of the compliant layer 38 that are in contact with and constrained by the vessel wall 44.

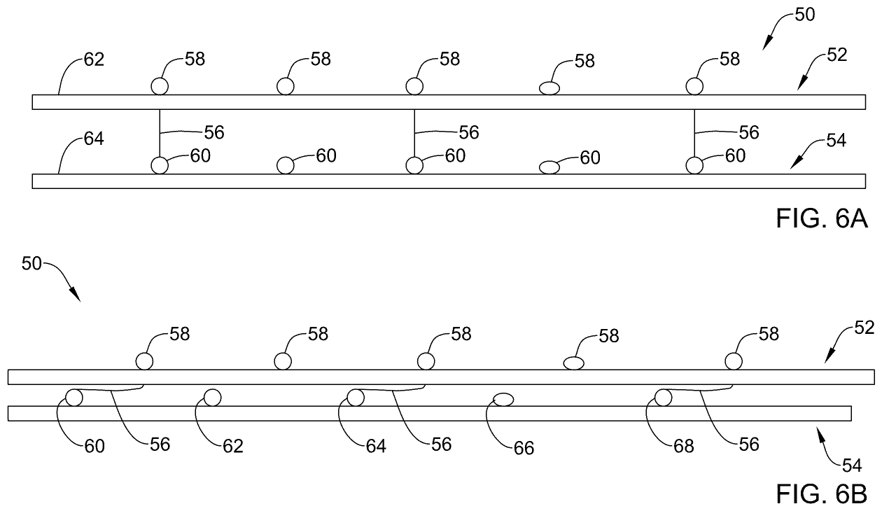

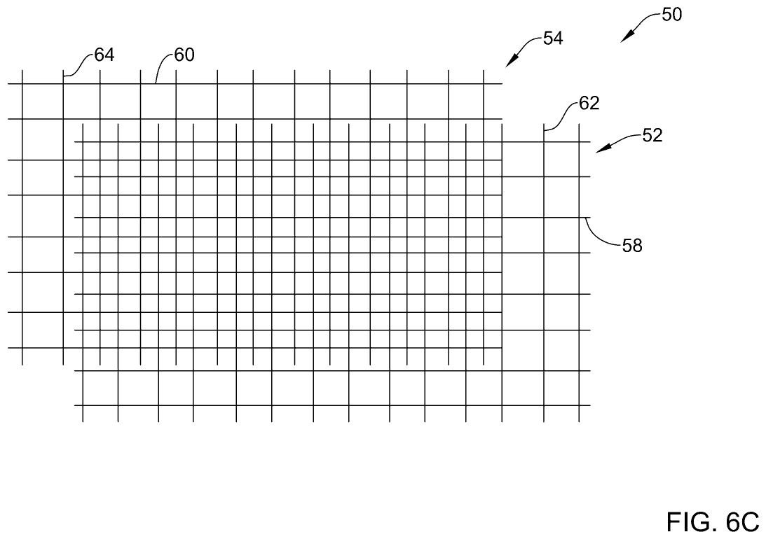

FIGS. 6A through 6C are schematic views of a portion of an example stent 50. The example stent 50 may be considered as being an example of the medical stent 20. The stent 50 includes an outer layer 52 and an inner layer 54. In some cases, the outer layer 52 may be considered as being a compliant layer while the inner layer 54 may be considered as being part of a tubular support. The outer layer 52 is coupled with the inner layer 54 via a number of struts 56 that extend between cross-members 58 forming part of the outer layer 52 and cross-members 60 forming part of the inner layer 54. The outer layer 52 may be considered as including additional cross-members 62 that extend transverse to the cross-members 58 and cross over and/or under the cross-members 58. The inner layer 54 may be considered as including additional cross-members 64 that extend transverse to the cross-members 60 and cross over and/or under the cross-members 60. The cross-members 56 and 62 may each be considered as being within a plane defined by the outer layer 52, and the cross-members 58 and 64 may each be considered as being within a plane defined by the inner layer 54. The cross-members 58 and 60 may be considered as extending into and above the plane of the drawing. In some instances, the cross-members 58 and 62 may be formed into a braided configuration in which the cross-members 62 are interwoven with the cross-members 58, forming the outer layer 52. Similarly, in some instances, the cross-members 60 and 64 may be formed into a braided configuration in which the cross-members 60 are interwoven with the cross-members 64, forming the inner layer 54. The medical stent 50 may be considered as an uncovered stent having passageways extending through the tubular wall of the medical stent 50 from an exterior of the medical stent 50 into the lumen of the medical stent 50. The passageways may be defined by the void spaces extending through the wall of the medical stent 50.

As shown in FIG. 6A, the outer layer 52 is at a relaxed configuration relative to the inner layer 54, which defines a first distance between the outer layer 52 and the inner layer 54. This may be considered as defining a first pore or void size within the stent 50. As shown in FIG. 6B, the outer layer 52 has moved closer to the inner layer 54, and in fact has shifted axially in the process, reducing the radial distance between the outer layer 52 and the inner layer 54, and thus reducing the radial thickness of the stent 50. While the struts 56 are seen to be orthogonal or at least substantially orthogonal (defined as within 10 percent of orthogonal) to the outer layer 52 and the inner layer 54 in FIG. 6A, the struts 56 are now parallel or at least substantially parallel (defined as within 10 percent of parallel) to the outer layer 52 and the inner layer 54 in FIG. 6B. This corresponds to the outer layer 52 being pushed closer to the inner layer 54 as a result of being constrained by contact with a blood vessel wall, for example, thereby reducing the radial distance between the outer layer 52 and the inner layer 54, and thus reducing the radial thickness of the stent 50.

FIG. 6C provides a schematic example of how the voids formed between the outer layer 52 and the inner layer 54, and the struts 56, may vary depending on the relative radial position of the outer layer 52 and the inner layer 54. As can be seen, the outer layer 52 includes a number of orthogonal cross-members 58 and 62 and the inner layer 54 includes a number of orthogonal cross-members 60 and 64. When the outer layer 52 and the inner layer 54 are positioned as shown in FIG. 6A, the cross-members 60 and 64 align with the cross-members 58 and 62, and the void size corresponds to a square defined by the distance between adjacent cross-members 58 and adjacent cross-members 62 (in the outer layer 52) or the distance between adjacent cross-members 60 and adjacent cross-members 64 (in the inner layer 54). When the outer layer 52 and the inner layer 54 are positioned as shown in FIG. 6B (and in FIG. 6C), and the outer layer 52 has shifted laterally (and radially) relative to the inner layer 54, the cross-members 60 and 64 do not align with the cross-members 58 and 62, and instead the cross-members 60 and 64 are instead positioned intermediate the cross-members 58 and 62 such that the cross-members 58 and 62 of the outer layer 52 transect or extend across the voids (e.g., open cells) of the inner layer 54, and effectively divide the voids (e.g., open cells) defined by the cross-members 60 and 64. As seen in FIG. 6C, the result is that the void size defined through the wall of the stent 50 are roughly twenty five percent that of the void size as shown in FIG. 6A. In other instances, the configuration of the outer layer 52 and the inner layer 54 may be such that radially constraining the outer layer 52 toward the inner layer 54 may reduce the effective void size defined through the wall of the stent 50 by 2 time or more, 3 times or more, 4 times or more, or 5 times or more, for example.

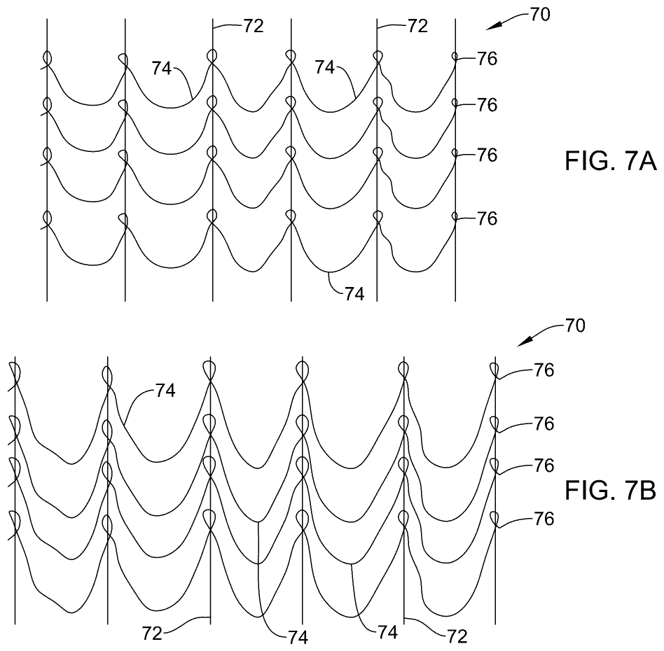

FIGS. 7A and 7B are schematic views of a portion of an example stent 70. The example stent 70 includes a number of struts 72. In some instances, the struts 72 may be part of a monolithic tubular structure, such as a laser-cut tubular structure. In some instances, the struts 72 may be part of a braided tubular structure. A number of out-of-plane loops 74 are formed by weaving a number of filaments 76 between the struts 72. The loops 74 may be formed in any of a variety of ways, including an open loop knitting pattern or a twisted loop knitting pattern. The medical stent 70 may be considered as an uncovered stent having passageways extending through the tubular wall of the medical stent 70 from an exterior of the medical stent 70 into the lumen of the medical stent 70. The passageways may be defined by the interstitial spaces or voids between the loops 74. In FIG. 7A, the loops 74 may be considered as extending upward out of a plane extending through the struts 72. This corresponds to a relaxed configuration in which the struts 72 and the loops 74 together define relatively larger voids that more easily allow blood to flow through. In FIG. 7B, the loops 74 may be considered as being bent or folded over relative to their position in FIG. 7A, thus reducing the radial thickness of the stent 70. In FIG. 7B, when the loops 74 are radially constrained and thus move closer to the struts 72, the loops 74 do not extend as far above the plane extending through the struts 72. As a result, the struts 72 and the loops 74 define relatively smaller voids through the wall of the stent 70 that are better at capturing plaque.

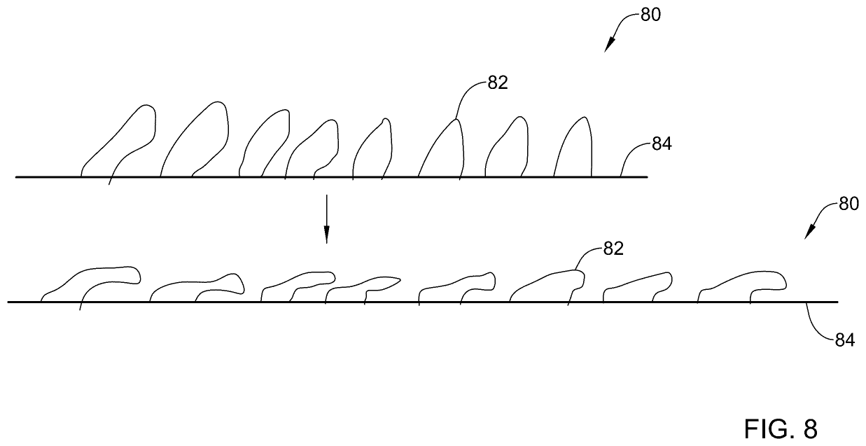

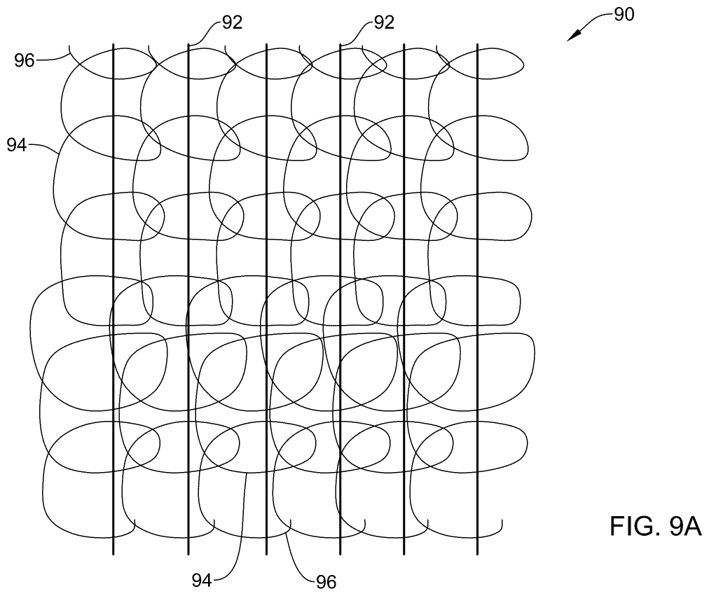

FIG. 8 is a schematic view of a portion of an example stent 80. The example stent 80 includes a number of woven loops 82 that are secured relative to a substrate 84. The substrate 84 may represent part of a stent, for example. The woven loops 82 may be formed in any of a variety of ways, including an open loop knitting pattern or a twisted loop knitting pattern. The medical stent 80 may be considered as an uncovered stent having passageways extending through the tubular wall of the medical stent 80 from an exterior of the medical stent 80 into the lumen of the medical stent 80. The passageways may be defined by the interstitial spaces or voids between the loops 82. At the top of FIG. 8, the woven loops 82 extend above the substrate 84, and may be considered as extending radially outwardly from the substrate 84, providing the stent 80 with a radial thickness. This corresponds to a relaxed configuration in which the woven loops 82 define relatively larger voids that more easily allow blood to flow through the wall of the stent 80. At the bottom of FIG. 8, the woven loops 82 have been compressed downward, toward the substrate 84, as a result of being constrained by contact with a vessel wall. The woven loops 82 may be considered as being bent or folded over, and do not extend as far in a radial direction relative to the substrate 84, reducing the radial thickness of the wall of the stent 80. As a result, the woven loops 82 define relatively smaller voids through the wall of the stent 80 that are better at capturing plaque.

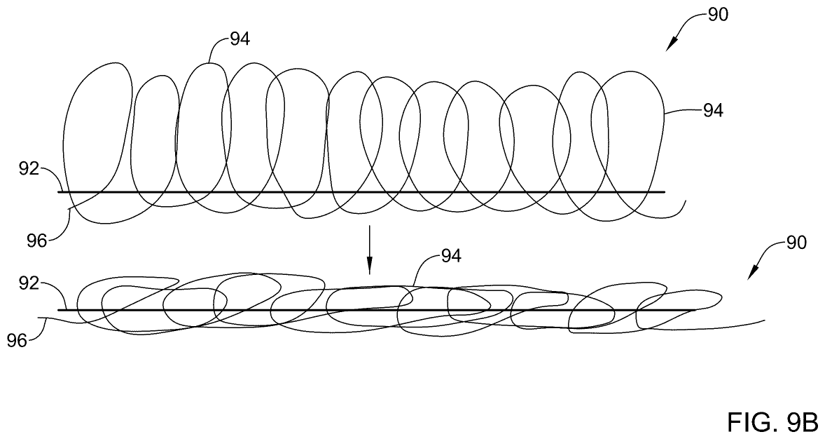

FIGS. 9A and 9B are schematic views of a portion of an example stent 90. FIG. 9A is a top view while FIG. 9B is a side view. The example stent 90 includes a number of stent struts 92 that as shown, are arranged parallel to each other. In some instances, the stent struts 92 may be part of a monolithic tubular structure, such as a laser-cut stent. In some instances, the stent struts 92 may be part of a braided stent. A number of coils 94 are wound around the stent struts 92, each of the coils 94 formed from a filament 96. The filaments 96 may be formed of any metal, for example. In some instances, there is a coil 94 wound around each of the stent struts 92. In some instances, each coil 94 may be wound around two or more adjacent stent struts 92. In some instances, a first stent strut 92 may include a coil 94, but an adjacent stent strut 92 may not. The stent struts 92 may alternate between including a coil 94 and not including a coil 94. In some instances, the coils 94 may be periodically welded or adhesively secured to the stent struts 92. The medical stent 90 may be considered as an uncovered stent having passageways extending through the tubular wall of the medical stent 90 from an exterior of the medical stent 90 into the lumen of the medical stent 90. The passageways may be defined by the interstitial spaces or voids between the coils 94.

In FIG. 9B, the top view shows the stent 90 in a relaxed configuration in which the coil 94 extends radially outwardly from the stent strut 92, providing the stent 90 with a relaxed radial wall thickness. The relaxed configuration defines relatively larger voids through the wall of the stent 90 that more easily allow blood to flow through. The bottom view in FIG. 9B shows the coil 94 in a radially compressed configuration as a result of the coil 94 opposing a vessel wall. As can be seen, the coil 94 has collapsed down on itself, reducing the radial thickness of the wall of the stent 90, and thereby forming relatively smaller voids through the wall of the stent 90 that may more easily capture plaque.

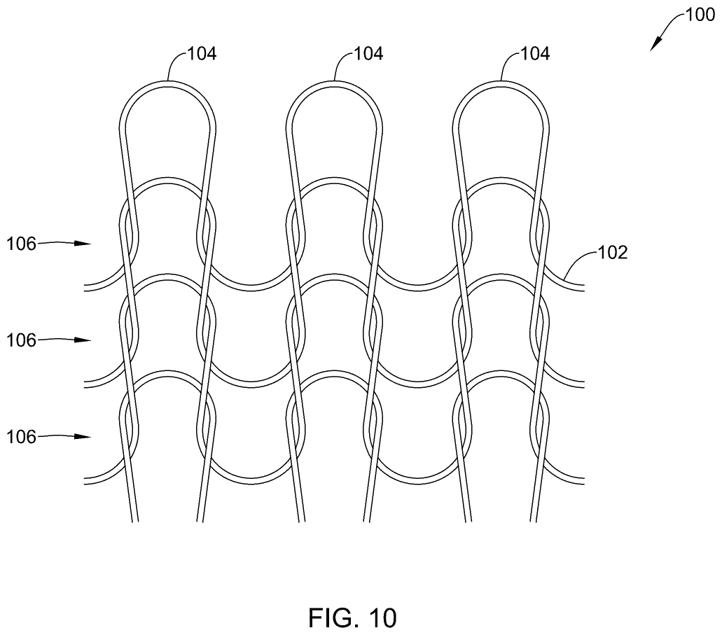

FIG. 10 is a schematic view of an example knitting pattern 100 that may be considered as an example of an open loop knitting pattern. The knitting pattern 100 may be used in forming the compliant layer 38 shown in some of the previous drawings. In some instances, the knitting pattern 100 may be used to form a stent that includes the compliant layer 38. The knitting pattern 100 may be produced using an automated weft knitting process that knits a filament 102 into parallel columns 104 and rows 106 of knit stitches. In some instances, the parallel columns 104 may run parallel to the longitudinal axis LA of the stent in both the expanded, relaxed configuration and an elongated, constrained configuration. At least some of the loops within the knitting pattern 100 may be adapted to extend radially outwardly, for example.

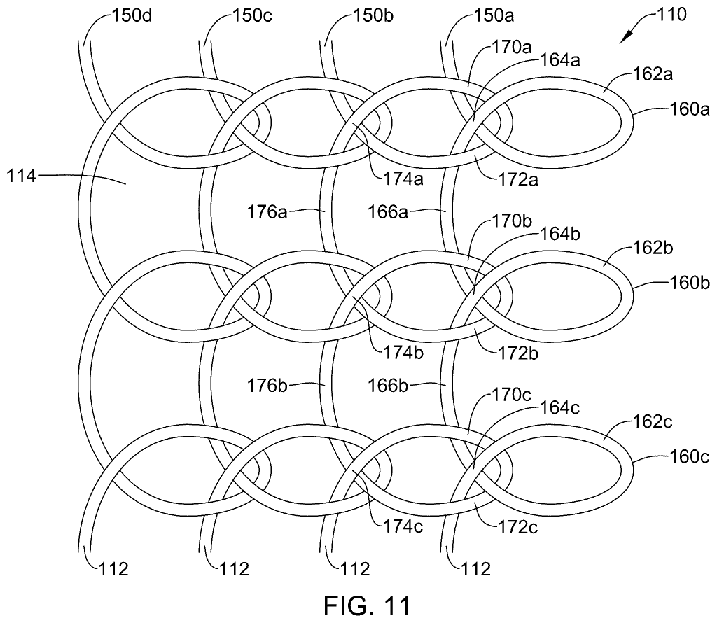

FIG. 11 is a schematic view of an example knitting pattern 110 that may be considered as an example of a twisted loop knitting pattern. The knitting pattern 110 may be used in forming the compliant layer 38 shown in some of the previous drawings. In some instances, the knitting pattern 110 may be used to form a stent that includes the compliant layer 38. The knitting pattern 110 is knitted from a filament 112 that is interwoven with itself, defining open cells 114. The filament 112 may be a monofilament. In some instances, the filament 112 may be two or more filaments wound, braided, or woven together.

The knitting pattern 110 may include a plurality of rows 150a, 150b, 150c, 150d (collectively, 150). The knitting pattern 110 may include any number of rows 150 desired. For example, the number of rows 150 may be selected to achieve a desired length. The uppermost, or first, row 150a may be unsecured and active. In some instances, the first row 150a may include a plurality of loops 160a, 160b, 160c (collectively, 160). The loops 160 may each include a loop portion 162a, 162b, 162c (collectively, 162) and an overlapping base portion 164a, 164b, 164c (collectively, 164). The overlapping base portion 164a, 164b, 164c is understood as the portion of the loops 160 in which one segment of the filament overlaps or crosses over a second segment of the filament, with the segment of the filament forming the loop portion 162a, 162b, 162c extending therebetween. Adjacent loops 160 may be interconnected by a rung section 166a, 166b (collectively, 166). For example, a first rung section 166a may extend between the base portion 164a of the first loop 160a and the second base portion 164b of the second loop 160b. The next row 150b may be suspended from the loops 160 of the first row 150a. For example, the second row 150b may include a plurality of loops 170a, 170b, 170c (collectively, 170) each including a loop portion 172a, 172b, 172c (collectively, 172) and a base portion 174a, 174b, 174c (collectively, 174). Adjacent loops 170 may be interconnected by a rung section 176a, 176b (collectively, 176). As the knitting pattern 110 is knitted, the loop portion 172 may be wrapped about the base portion 164 of the preceding row 150a.

It is contemplated that a single row 150 may be formed at a time. For example, the rows may be formed in succession with a subsequent row (e.g., row 150b) being formed after the preceding row (e.g., row 150a) has formed a complete rotation. While not explicitly shown, the loops 160 of the first row 150a may be wrapped about a section of the filaments 112 free from loops. As described herein, the loops 170 of the second row 150b may be wrapped about the base portion 164 of the loops 160 the preceding row 150a. For example, the filament 112 may be knitted such that it extends from the first rung section 176a, is wrapped about the base portion 164b of the preceding row 150a, crosses back over itself to form base section 174b and continues to the next rung section 176b. It is contemplated that the loop portion 170 may be positioned on a first side of the rungs 166a, 166b and on a second opposite side of the loop portion 162b. In other words, the filament 112 may be wound such that it extends on top of the second rung portion 166b, behind the base portion 164b, and over the first rung portion 166a before crossing over itself to form the base portion 174b of the loop 170b of the second row 150b. The reverse configuration is also contemplated in which the filament 112 may be wound such that it extends behind the second rung portion 166b, over or on top of the base portion 164b, and behind the first rung portion 1166a before crossing over itself to form the base portion 174b of the loop 170b of the second row 150b. At least some of the loops within the knitting pattern 110 may be adapted to extend radially outwardly, for example.

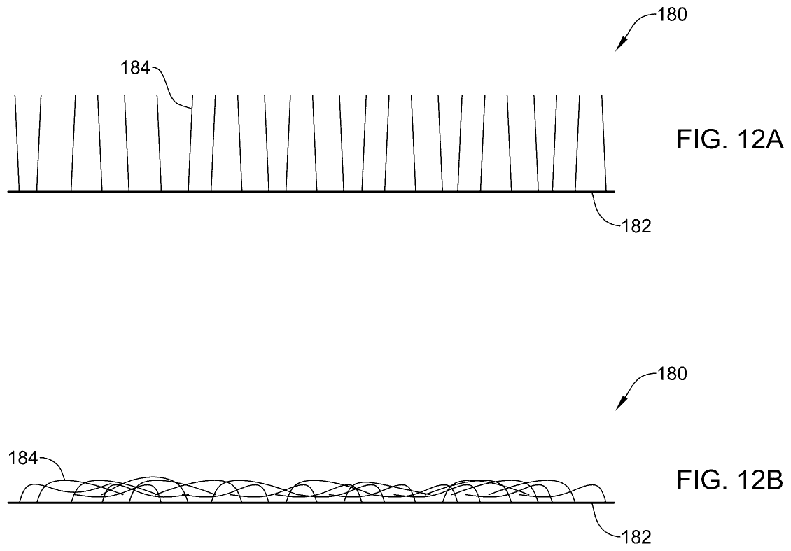

FIGS. 12A and 12B are schematic views of a portion of an example stent 180. The example stent 180 may include a number of struts 182 forming a tubular substrate, although only one strut 182 is shown. In some instances, the struts 182 may be part of a monolithic tubular structure, such as a laser-cut tubular structure. In some instances, the struts 182 may be part of a braided structure. A number of strands 184 are secured to the strut 182 and extend radially outwardly from the strut 182. In some instances, the strands 184 may be adhesively secured to the strut 182. Other attachment methods such as welding or soldering may also be used. The medical stent 180 may be considered as an uncovered stent having passageways extending through the tubular wall of the medical stent 180 from an exterior of the medical stent 180 into the lumen of the medical stent 180. The passageways may be defined by the interconnected interstitial spaces or voids between the strands 184 and struts 182. As seen in FIG. 12A, the strands 184 extending radially outwardly from the strut 182 may be considered as a relaxed configuration in which the struts 182 and the strands 184 together define a radially wall thickness of the stent 180 having relatively larger voids that more casily allow blood to flow through the wall of the stent 180. In FIG. 12B, the strands 184 have been bent or folded over relative to their position in FIG. 12A, thereby reducing the radial wall thickness of the stent 180. In FIG. 12B, the strands 184 do not extend as far above the strut 182, and as a result the struts 182 and the strands 184 define relatively smaller voids through the wall of the stent 180 that are better at capturing plaque.

The materials that can be used for the various components of the medical stent(s), and the various elements thereof disclosed herein may include those commonly associated with medical devices. For simplicity purposes, the following discussion refers to the apparatus. However, this is not intended to limit the devices and methods described herein, as the discussion may be applied to other elements, members, components, or devices disclosed herein, such as, but not limited to, the medical stent and/or elements or components thereof. In some instances, the apparatus, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-clastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some instances, portions or all of the apparatus, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the apparatus in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the apparatus to achieve the same result.

In some instances, a degree of Magnetic Resonance Imaging (MRI) compatibility is imparted into the apparatus and/or other elements disclosed herein. For example, the apparatus, and/or components or portions thereof, may be made of a material that does not substantially distort the image and create substantial artifacts (e.g., gaps in the image). Certain ferromagnetic materials, for example, may not be suitable because they may create artifacts in an MRI image. The apparatus, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some instances, the apparatus and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

Having thus described several illustrative examples of the present disclosure, those of skill in the art will readily appreciate that yet other examples may be made and used within the scope of the claims hereto attached. It will be understood, however, that this disclosure is, in many respects, only illustrative. Changes may be made in details, particularly in matters of shape, size, arrangement of parts, and exclusion and order of steps, without exceeding the scope of the disclosure. The disclosure's scope is, of course, defined in the language in which the appended claims are expressed.

What is claimed is:

1. A medical stent, comprising:
an expandable structure moveable between a collapsed configuration for delivery and an expanded configuration for deployment, the expandable structure in the expanded configuration defining a relaxed outer diameter;
wherein:
the expandable structure retains its relaxed outer diameter along portions of the expandable structure that are not constrained by contact with vessel walls after deployment; and
the expandable structure moves to a reduced outer diameter less than the relaxed outer diameter along portions of the expandable structure that are compressed by contact with vessel walls after deployment.

2. The medical stent of claim 1, wherein the expandable structure defines a plurality of voids through a wall of the expandable structure, and wherein:
the voids have a first average size when the expandable structure retains its relaxed configuration; and
the voids have a second average size less than the first average size when the expandable structure is compressed to the reduced outer diameter.

3. The medical stent of claim 1, wherein the expandable structure comprises an inner layer and an outer layer moveable relative to the inner layer, and wherein:
the outer layer is spaced a first distance from the inner layer when the expandable structure retains its relaxed outer diameter; and
the outer layer is spaced a second distance from the inner layer when the expandable structure is compressed to its reduced outer diameter;
wherein the second distance is less than the first distance.

4. The medical stent of claim 1, wherein the expandable structure comprises a knitted stent.

5. The medical stent of claim 4, wherein the knitted stent comprises a plurality of loops, at least some of which have a relaxed configuration in which the loops extend radially outwardly relative to a longitudinal axis of the medical stent.

6. The medical stent of claim 5, wherein the loops that extend radially outwardly relative to the longitudinal axis of the medical stent are adapted to fold over in response to being compressed by contact with the vessel wall.

7. The medical stent of claim 5, wherein the loops having a relaxed configuration in which the loops extend radially outwardly define the relaxed outer diameter of the expandable structure.

8. The medical stent of claim 6, wherein the loops that are adapted to fold over in response to being compressed define the reduced outer diameter of the expandable structure when the loops are folded over.

9. The medical stent of claim 1, wherein the expandable structure comprises:
one or more elongated struts; and
one or more filaments wrapped around each of the one or more elongated struts;
wherein:
the one or more filaments form coils having a relaxed configuration defining the relaxed outer diameter of the expandable structure; and
portions of the coils that are compressed from contact with the vessel wall define the reduced outer diameter of the expandable structure.

10. A medical stent, comprising:
a tubular substrate extending from a proximal end to a distal end, the tubular substrate moveable between a radially collapsed configuration and a radially expanded configuration; and
a compliant layer extending over at least part of the tubular substrate, the compliant layer having a relaxed thickness when not radially constrained, the compliant layer adapted to radially compress into a reduced thickness when radially constrained.

11. The medical stent of claim 10, wherein the compliant layer defines a plurality of voids, and wherein:
the voids have a first average size when the compliant layer retains its relaxed configuration; and
the voids have a second average size less than the first average size when the compliant layer is compressed to the reduced thickness.

12. The medical stent of claim 10, wherein the tubular substrate comprises an inner layer and the compliant layer comprises an outer layer moveable relative to the inner layer, and wherein:
the outer layer is spaced a first distance from the inner layer when the compliant layer retains its relaxed thickness; and
the outer layer is spaced a second distance from the inner layer when the compliant layer is compressed to its reduced thickness;
wherein the second thickness is less than the first thickness.

13. The medical stent of claim 10, wherein the compliant layer comprises a plurality of loops, at least some of which have a relaxed configuration in which the loops extend radially outwardly relative to a longitudinal axis of the medical stent.

14. The medical stent of claim 13, wherein the loops that extend radially outwardly relative to the longitudinal axis of the medical stent are adapted to fold over in response to being radially compressed by contact with the vessel wall.

15. The medical stent of claim 13, wherein the loops having a relaxed configuration in which the loops extend radially outwardly define the relaxed thickness of the compliant layer.

16. The medical stent of claim 15, wherein the loops that are adapted to fold over in response to being radially compressed define the reduced thickness of the compliant layer when the loops are folded over.

17. A medical stent adapted for deployment within a blood vessel having a side branch extending from the blood vessel, the medical stent adapted to be deployed within the blood vessel at a position in which the medical stent spans the side branch, the medical stent comprising:
a substrate extending from a proximal end to a distal end, the substrate defining a tubular body moveable between a radially collapsed configuration and a radially expanded configuration; and
a compliant layer extending over at least part of the substrate, the compliant layer adapted to being radially compressed by contact with a vessel wall of the blood vessel;
wherein a first portion of the compliant layer is configured to be radially compressed by contact with the vessel wall when deployed within the blood vessel; and
wherein a second portion of the compliant layer corresponding to where the side branch extends is configured to not be radially compressed or be compressed to a lesser degree than the first portion when deployed within the blood vessel.

18. The medical stent of claim 17, wherein the first portion of the compliant layer has a smaller void size than that of the second portion of the compliant layer.

19. The medical stent of claim 17, wherein the first portion of the compliant layer holds plaque in place between the compliant layer and the vessel wall.

20. The medical stent of claim 17, wherein the second portion of the compliant layer allows blood to flow through the wall of the medical stent between the blood vessel and the side branch.
